(19) United States
(12) Patent Application Publication
(10) Pub. No.: US 2003/0216366 A1
(43) Pub. Date: Nov. 20, 2003

(54) Step-down estrogen therapy

(76) Inventors: Thomas W. Leonard, Wilmington, NC (US); R. Forrest Waldon, Wilmington, NC (US)

Correspondence Address: MYERS BIGEL SIBLEY & SAJOVEC, PO BOX 37428, RALEIGH, NC, 27627, US

(21) Appl. No.: 10/356,242

(22) Filed: Jan. 31, 2003

Related U.S. Application Data
(60) Provisional application No. 60/369,905, filed on Apr. 3, 2002.

(51) Int. Cl. A61K 31/56

(52) U.S. Cl. 514/182

(57) ABSTRACT

The present invention includes methods for treating vasomotor symptoms through the administration of estrogenic compounds. The methods presented may include starting estrogen therapy at a high dose, and then lowering the dose once therapy has been shown to be effective.

### CROSS REFERENCE TO RELATED APPLICATIONS

This application claims priority from U.S. Provisional Patent Application No. 60/369,905, filed Apr. 3, 2002, the disclosure of which is incorporated herein by reference in its entirety.

### FIELD OF THE INVENTION

The present invention generally relates to a method of treating hormonal deficiencies in women, particularly menopausal and post-menopausal women.

### BACKGROUND OF THE INVENTION

Menopause typically occurs in women during middle age and is often described as an ovarian shutdown. Menopause is usually associated with a profound decrease in circulating levels of estrogens. Currently, there are a large variety of disorders and conditions that are attributed to the reduction of estrogen levels. These disorders and conditions include hot flashes, dryness and atrophy of the vagina, parathesia, dyspareunia, osteoporosis, and an increase in cardiovascular disease. In an effort to reduce these disorders and conditions, estrogens are administered to women in a so-called “estrogen replacement therapy”. Estrogen replacement therapy continues to be the primary treatment of such disorders and conditions associated with menopause. Additionally, estrogens may also be used in postmenopausal women in the treatment of osteoporosis and to delay onset of or prevent cardiovascular disease and Alzheimer's.

One of the risks associated with the administration of estrogen replacement therapy is that women with intact uteri may develop endometrial hyperplasia. The term “endometrial hyperplasia” refers to the over stimulation of the lining of the uterus, which is a precursor to endometrial or uterine cancer. The development of endometrial hyperplasia is a significant issue with estrogen replacement therapy. For example, it has been observed in U.S. Pat. No. RE 36,247 to Plunkett, et al., and U.S. Pat. No. 5,043,331 to Hirvonen, that the co-administration of progestin can blunt the effect of estrogens. However, side effects often still occur with this co-administration. Thus, it would be desirable to have an estrogen replacement therapy in which the potential side effects relating to such therapy were reduced.

Presently, the labeling of currently marketed estrogen products and the FDA's own “Labeling Guidance for Non-Contraceptive Estrogen Drug Products” recommends the lowest dose and regimen that will control vasomotor symptoms. However, administration of the lowest dose to begin estrogen replacement therapy often does not treat a wide range in the severity of vasomotor symptoms. Thus it may be desirable to relieve vasomotor symptoms through alternative methods of estrogen therapy.

### SUMMARY OF THE INVENTION

The present invention discloses methods of treating vasomotor symptoms and menopause. The present invention also discloses various methods of estrogen therapy and hormonal replacement therapy. The methods employed by the present invention include administering a higher first dose of an estrogenic compound in estrogen therapy followed by treatment with a secondary dose of an estrogenic compound after a reduction in the vasomotor symptoms has been effectively established.

### BRIEF DESCRIPTION OF THE DRAWINGS

FIG. 1 is a graph illustrating the percent reduction in mean frequency of moderate to severe hot flushes in a subject undergoing estrogen therapy.

FIG. 2 is a graph depicting the percent reduction in mean severity of vasomotor symptoms in a subject undergoing estrogen therapy.

### DETAILED DESCRIPTION OF THE EMBODIMENTS

The invention will now be described with reference to the embodiments set forth herein. These embodiments are intended to illustrate the invention and are not meant to limit the scope of the invention.

In one aspect, the invention relates to a method of administering a pharmaceutical composition. The pharmaceutical composition comprises a therapeutically effective amount of an estrogenic compound, and a pharmaceutically acceptable carrier. The composition may also contain an androgenic compound, wherein the androgenic compound is preferably a non-aromatizing androgen. Additionally, the composition may contain a progestational agent.

A “therapeutically effective” amount as used herein is an amount of an estrogenic compound that is sufficient to treat hormonal deficiencies in a subject. The therapeutically effective amount will vary with the age and physical condition of the patient, the severity of the treatment, the duration of the treatment, the nature of any concurrent treatment, the pharmaceutically acceptable carrier used and like factors within the knowledge and expertise of those skilled in the art. Pharmaceutically acceptable carriers are preferably solid dosage forms such as tablets or capsules. Liquid preparations for oral administration may be also be used and may be prepared in the form of syrups or suspensions, e.g., solutions containing an active ingredient, sugar, and a mixture of ethanol, water, glycerol, and propylene glycol. If desired, such liquid preparations may contain coloring agents, flavoring agents, and saccharin. Thickening agents such as carboxymethylcellulose may also be used. Additionally, transdermal patches and other acceptable carriers, the selection of which are known in the art.

Estrogen levels are related to the general physiological health of postmenopausal women. They exert positive central nervous system (CNS) effects on hot flashes, and improve nerve transmission which is believed to delay various types of dementia. They have positive cardiovascular effects by improving lipid levels and promoting vasodilation and relaxation. They also contribute to health of the vagina, provide local vasodilation effects and stimulate mucous production. Suitable estrogenic compounds include estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ8,9-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof, and the estrogen ketones and their corresponding 17α- and 17-β hydroxy derivatives. The estrogenic compounds may also be present as conjugated estrogens. Approximately 1.0 mg of 17β-estradiol is equivalent to 0.625 mg of conjugated estrogens. The conjugates may be various conjugates understood by those skilled in the art, including, but not limited to, sulfate and glucuronide. The most preferred estrogen conjugates are estrogen sulfates. The estrogenic compounds may also be present as salts of estrogens conjugates. The salts may be various salts understood by those skilled in the art, including, but not limited to, sodium salts, calcium salts, magnesium salts, lithium salts, and piperazine salt. The most preferred salts are sodium salts. The estrogenic compounds can be derived from natural and synthetic sources. Preferably, the therapeutically effective amount estrogenic compound is about 0.05 to about 3 mg, and preferably about 0.5 to about 2 mg based on oral dose equivalents of estradiol.

As previously stated, androgenic compounds may be combined with the estrogenic compounds. Suitable androgenic compounds include both aromatizing and non-aromatizing compounds. Non-aromatizing compounds include as oxandrolone, oxymetholone, stanozolol, stanozolone, danazol, pharmaceutically acceptable esters and salts thereof, and combinations of any of the foregoing. Aromatizing compounds included, but are not limited to, androsterone, androstenediol, 4-androstene-3, 17-dione, and (3α, 5α)-androst-16-en-3-ol. Preferably, the therapeutically effective amount of the androgenic compound is about 0.25 to about 10 mg. For women suffering from androgen deficiency the oral dosage equivalents of oxandrolone is about 0.5 to 4 mg of an androgenic compound per day.

Additionally, as previously stated, a progestational agent may be used in combination with the estrogenic compound. Examples of progestational agents are set forth in U.S. Pat. No. Re. 36,247 to Plunkett et al. Examples include, but are not limited to, laevo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, and cyproterone acetate.

The estrogen formulations of the present invention may be, for example, in the form of tablets; effervescent tablets; pills; powders; elixirs; suspensions; emulsions; solutions; syrups; soft and hard gelatin capsules; transdermal patches; topical gels, creams and the like; vaginal suppositories; sterile injectable solutions; and sterile packaged powders, sublingual tablets, buccal tablets and buccal adhesive systems.

In certain embodiments, the drug product is present in a solid pharmaceutical composition that may be suitable for oral administration. A solid composition of matter according to the present invention may be formed and may be mixed with and/or diluted by an excipient. The solid composition of matter may also be enclosed within a carrier which may be, for example, in the form of a capsule, sachet, tablet, paper, or other container. When the excipient serves as a diluent, it may be a solid, semi-solid, or liquid material which acts as a vehicle, carrier, or medium for the composition of matter.

Various suitable excipients will be understood by those skilled in the art and may be found in the National Formulary, 19: 2404-2406 (2000), the disclosure of pages 2404 to 2406 being incorporated herein in their entirety. Examples of suitable excipients include, but are not limited to, starches, gum arabic, calcium silicate, microcrystalline cellulose, methacrylates, shellac, polyvinylpyrrolidone, cellulose, water, syrup, and methylcellulose. The drug product formulations can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to, mannitol, sorbitol, xylitol, sucrose, maltose, glucose, lactose, dextrose, and the like. Suitable buffers encompass, but are not limited to, phosphate, citrate, tartarate, succinate, and the like. Other inert fillers which may be used encompass those which are known in the art and are useful in the manufacture of various dosage forms. If desired, the solid formulations may include other components such as bulking agents and/or granulating agents, and the like. The drug products of the invention may be formulated so as to provide quick, sustained, or delayed release of the active ingredient after administration to the patient by employing procedures well known in the art.

To form tablets for oral administration, the composition of matter of the present invention may be made by a direct compression process. In this process, the active drug ingredients may be mixed with a solid, pulverant carrier such as, for example, lactose, saccharose, sorbitol, mannitol, starch, amylopectin, cellulose derivatives or gelatin, and mixtures thereof, as well as with an antifriction agent such as, for example, magnesium stearate, calcium stearate, and polyethylene glycol waxes. The mixture may then be pressed into tablets using a machine with the appropriate punches and dies to obtain the desired tablet size. The operating parameters of the machine may be selected by the skilled artisan. Alternatively, tablets for oral administration may be formed by a wet granulation process. Active drug ingredients may be mixed with excipients and/or diluents. The solid substances may be ground or sieved to a desired particle size. A binding agent may be added to the drug. The binding agent may be suspended and homogenized in a suitable solvent. The active ingredient and auxiliary agents may also be mixed with the binding agent solution. The resulting dry mixture is moistened with the solution uniformly. The moistening typically causes the particles to aggregate slightly, and the resulting mass is pressed through a stainless steel sieve having a desired size. The mixture is then dried in controlled drying units for the determined length of time necessary to achieve a desired particle size and consistency. The granules of the dried mixture are sieved to remove any powder. To this mixture, disintegrating, antifriction, and/or anti-adhesive agents are added. Finally, the mixture is pressed into tablets using a machine with the appropriate punches and dies to obtain the desired tablet size. The operating parameters of the machine may be selected by the skilled artisan.

If coated tablets are desired, the above prepared core may be coated with a concentrated solution of sugar or cellulosic polymers, which may contain gum arabic, gelatin, talc, titanium dioxide, or with a lacquer dissolved in a volatile organic solvent or a mixture of solvents. To this coating various dyes may be added in order to distinguish among tablets with different active compounds or with different amounts of the active compound present. In a particular embodiment, the active ingredient may be present in a core surrounded by one or more layers including enteric coating layers.

Soft gelatin capsules may be prepared in which capsules contain a mixture of the active ingredient and vegetable oil. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, and/or gelatin.

In one preferred embodiment, the formulation is in the form of orally-administered tablets which contain the composition of matter of the present invention as set forth herein along with the following inactive ingredients: calcium phosphate tribasic, calcium sulfate, carnauba wax, cellulose, glyceryl monooleate, lactose, magnesium stearate, methylcellulose, pharmaceutical glaze, polyethylene glycol, stearic acid, sucrose, and titanium dioxide. Such ingredients may be present in amounts similar to those present in Premarin® (conjugated estrogens tablets, USP) made commercially available by Wyeth-Ayerst Laboratories of Philadelphia, Pa. Tablets employing the active ingredients of the invention may contain excipients similar to those contained in the 0.3 mg, 0.625 mg, and 1.25 mg tablets of Premarin® (conjugated estrogens tablets, USP).

Liquid preparations for oral administration may be prepared in the form of syrups or suspensions, e.g., solutions containing an active ingredient, sugar, and a mixture of ethanol, water, glycerol, and propylene glycol. If desired, such liquid preparations may contain coloring agents, flavoring agents, and saccharin. Thickening agents such as carboxymethylcellulose may also be used.

In the event that the above formulations are to be used for parenteral administration, such a formulation may comprise sterile aqueous injection solutions, non-aqueous injection solutions, or both comprising the composition of matter of the present invention. When aqueous injection solutions are prepared, the composition of matter may be present as a water soluble pharmaceutically acceptable salt. Parenteral preparations may contain anti-oxidants, buffers, bacteriostats, and solutes which render the formulation isotonic with the blood of the intended recipient. Aqueous and non-aqueous sterile suspensions may include suspending agents and thickening agents. The formulations may be presented in unit-dose or multi-dose containers, for example sealed ampules and vials. Extemporaneous injection solutions and suspensions may be prepared from sterile powders, granules and tablets of the kind previously described.

In a preferred embodiment, the drug product of the present invention is in the form of an injectable solution containing a predetermined amount (e.g., 25 mg) of the composition of matter in a sterile lyophilized cake which also contains lactose, sodium citrate, and simethicone. The pH of a solution containing the above ingredients may be adjusted using a suitable buffer (e.g., sodium hydroxide or hydrochloric acid). Reconstitution may be carried out according to known methods, e.g., using a sterile diluent (5 mL) containing 2 percent by volume benzyl alcohol in sterile water. A preferred injectable solution is similar to Premarin® Intravenous made commercially available by Wyeth-Ayerst Laboratories.

The composition of matter also may be formulated such that it may be suitable for topical administration (e.g., vaginal cream). These formulations may contain various excipients known to those skilled in the art. Suitable excipients may include, but are not limited to, cetyl esters wax, cetyl alcohol, white wax, glyceryl monostearate, propylene glycol, monostearate, methyl stearate, benzyl alcohol, sodium lauryl sulfate, glycerin, mineral oil, water, carbomer, ethyl alcohol, acrylate adhesives, polyisobutylene adhesives, and silicone adhesives.

The drug product may be in the form- of a vaginal cream containing the composition of matter as set forth herein present in a nonliquefying base. The nonliquefying base may contain various inactive ingredients such as, for example, cetyl esters wax, cetyl alcohol, white wax, glyceryl monostearate, propylene glycol, monostearate, methyl stearate, benzyl alcohol, sodium lauryl sulfate, glycerin, and mineral oil. Such composition may be formulated similar to Premarin® Vaginal Cream made commercially available by Wyeth-Ayerst Laboratories.

Dosage units for vaginal or rectal administration may be prepared in the form of suppositories which may contain the composition of matter in a mixture with a neutral fat base polyethylene glycol, or they may be prepared in the form of gelatin-rectal capsules which contain the active substance in a mixture with a vegetable oil or paraffin oil.

In a preferred embodiment of the present invention, an estrogenic compound comprising 0.05 to 3 mg of estrogens, preferably conjugated estrogens, may be administered to a subject. The estrogenic compound may be used to treat vasomotor symptoms including, but not limited to hot flashes, also known as hot flushes, cold flashes/flushes, night and day sweats, dry vagina, dry hair and skin, insomnia, bladder problems and moodiness. The estrogenic compound may also be used to treat menopause or may be used in conjunction with or as an estrogen replacement therapy or hormonal replacement therapy.

The methods used in the present invention may include reducing the amount of an estrogen given to a subject by starting out administering a high dose of an estrogenic compound to a subject and then gradually lowering the dose once therapy has been effectively established. One skilled in the art will be able to use a number of permutations in which the dosage of the estrogenic compound may be lowered.

As stated above, the methods used in estrogen therapy in the present invention may include starting estrogen therapy at a high dose, and then lowering the dose once therapy has been shown to be effective. Preferably, the estrogenic compound is administered in a therapeutic amount to a subject in a first dose is sufficient to alleviate vasomotor symptoms. The first dose may be administered daily, continuously and uninterruptedly for an effective time period until such time that therapy has been effectively established, preferably two to twelve weeks, more preferably four to eight weeks. The therapeutic amount of the estrogenic compound for the first dose is typically 0.05 to 3 mg of estrogens, more preferably 0.6 to 1.25 mg of estrogens. After the cycle for the therapeutic amount of an estrogenic compound of a first dose is completed, a second dose of a therapeutic amount of an estrogenic compound is administered to a subject. This second dose comprises a lower dosage of the therapeutic amount of the estrogenic compounds than the first dose. Preferably, the therapeutic amount of the estrogenic compound in the second dose is 0.05 to 2.5 mg, and more preferably 0.25 to 0.5 mg per dose. The second dose is administered continuously and uninterruptedly until a time when all vasomotor symptoms and other symptoms relating to menopause have been alleviated and will not return.

Additionally, once therapy has been effectively established it may be possible to continue the step-down therapy as disclosed above by decreasing the amount of estrogenic compound in a third or fourth dose. One skilled in the art will be able to choose additional regimens based upon this information.

The present invention is explained in greater detail in the Examples which follow. These examples are intended as illustrative of the invention and are not to be taken are limiting thereof.

### EXAMPLES

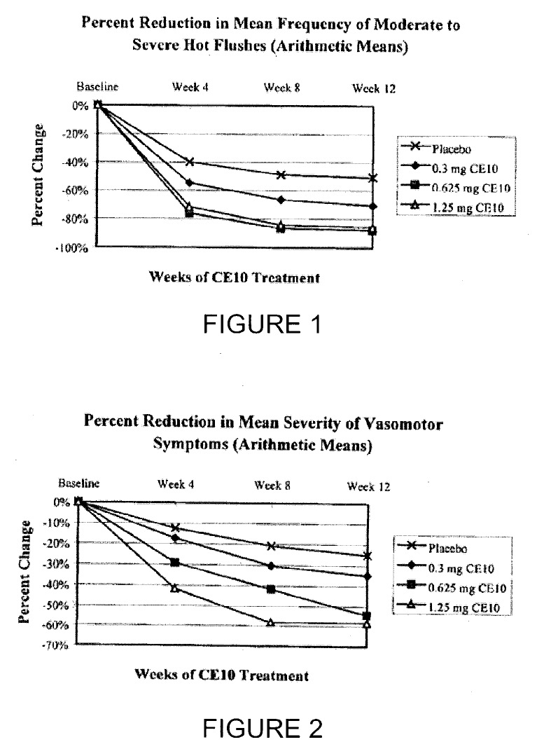

In a study conducted by the applicants, 281 post-menopausal women were randomly assigned to receive 0.3 mg, 0.625 mg, 1.25 mg of conjugated estrogens or a placebo. The post-menopausal women in the study were required to have at least fifty moderate to severe hot flashes per week or seven per day during the two week baseline assessment period. All of the subjects whom were administered conjugated estrogens exhibited a reduction in the amount of hot flashes that was greater than the subjects who were administered a placebo. Additionally, the subjects who received the administration of a conjugated estrogen dose exhibited a reduction in the severity of vasomotor symptoms. Thus, despite the placebo response as exhibited in FIG. 1, subjects receiving either 0.625 mg or 1.25 mg of conjugated estrogens showed signs of statistically significantly fewer moderate to severe hot flashes at weeks 4, 8 and 12 as compared with women receiving the placebo. The data from FIG. 1 demonstrates a maximal effect in the reduction of moderate to severe hot flashes within eight weeks. For the 0.3 mg conjugated estrogen dose, clinically significant results were seen at week 4. Achievement of a plateau for the efficacy of the 0.3 mg dose was observed after 12 weeks. Statistically, significant improvement was seen with the 0.3 mg dose of conjugated estrogens in the reduction of the frequency of moderate to severe hot flashes at weeks 8 and 12.

FIG. 2 illustrates the percent reduction in mean severity of vasomotor symptoms as a function of time for the placebo, 0.3, 0.625 and 1.25 doses of conjugated estrogens. The graph shows that for the 1.25 mg conjugated ester group, a maximal effect in the reduction of the severity of vasomotor symptoms was demonstrated within eight weeks. For the 0.625 mg dose, the effects at the 12 week period were remarkably similar to the percent reduction as the 1.25 mg conjugated estrogen dose. Both the 0.625 and the 1.25 mg doses showed significant improvements as compared to the placebos given in weeks four, eight and twelve. For the 0.3 mg dose of conjugated estrogens, at week 4, a clinically significant result was observed for the improvement in vasomotor symptoms and there was an overall reduction in severity of the vasomotor symptoms over the entire twelve week treatment period. Thus, the 0.6 and 1.25 mg doses demonstrate estrogen replacement therapy that is more effective than the lesser dose.

In the specification, there has been disclosed typical preferred embodiments of the invention and, although specific terms are employed, they are used in a generic and descriptive sense only and not for purposes of limitation of the scope of the invention being set forth in the following claims.

What is claimed is:

1. A method of treating vasomotor symptoms comprising:
administering a first dose of a therapeutic amount of an estrogenic compound to a subject; and
administering a second dose of a therapeutic amount of an estrogenic compound at a later time period to the subject, said second dose comprising a lower dosage of said therapeutic amount of an estrogenic compound than said first dose.

2. The method according to claim 1, wherein said estrogenic compound is a conjugated estrogen.

3. The method according to claim 1, wherein said first dose comprises 0.5 to 3 mg of an estrogenic compound.

4. The method according to claim 1, wherein said first dose comprises 0.6 to 1.25 mg of an estrogenic compound.

5. The method according to claim 1, wherein said estrogenic compound is selected from the group consisting of estrone, 17α-estradiol, 17β-estradiol, equilin, 17α-dihydroequilin, 17β0-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

6. The method according to claim 1, further comprising administering a progestin in a daily dose.

7. The method according to claim 1, further comprising administering an androgen compound in a daily dose.

8. The method according to claim 7, wherein the androgenic compound is selected from the group consisting of oxandrolone, oxymetholone, stanozolol, stanozolone, danazol, pharmaceutically acceptable esters and salts thereof, and combinations of any of the foregoing.

9. The method according to claim 1, wherein said second dose of an estrogenic compound is administered after therapy of the vasomotor symptoms has been effectively established.

10. The method according to claim 1, wherein said second dose of an estrogenic compound is administered between 2 weeks and 12 weeks after the first dose of an estrogenic compound.

11. The method according to claim 1, wherein said second dose of an estrogenic compound is administered between 4 weeks and 8 weeks after the first dose of an estrogenic compound.

12. The method according to claim 1, wherein said vasomotor symptoms are selected from the group of hot flashes, cold flashes, night sweats, day sweats, dry vagina, dry hair and skin, insomnia, bladder problems and moodiness.

13. The method according claim 1, wherein said first dose is continuously and uninterruptedly administered to said subject for a predetermined period of time and then said second dose is continuously and uninterruptedly administered to said subject.

14. The method according to claim 1, comprising:
administering a third dose of a therapeutic amount of an estrogenic compound at a later time period to the subject than that of said second dose, said third dose comprising a lower dosage of said therapeutic amount of an estrogenic compound than said second dose.

15. The method according to claim 1, wherein said subject is human.

16. A method of treating menopause comprising:
administering a first dose of a therapeutic amount of an estrogenic compound to a subject; and
administering a second dose of a therapeutic amount of an estrogenic compound to the subject at a later time period, said second dose comprising a lower dosage of said therapeutic amount of an estrogenic compound than said first dose.

17. The method according to claim 16, wherein said estrogenic compound is a conjugated estrogen.

18. The method according to claim 16, wherein said first dose comprises 0.5 to 3 mg of an estrogenic compound.

19. The method according to claim 16, wherein said first dose comprises 0.6 to 1.25 mg of an estrogenic compound.

20. The method according to claim 16, wherein said estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

21. The method according to claim 16, further comprising administering a progestin in a daily dose.

22. The method according to claim 16, further comprising administering an androgen compound in a daily dose.

23. The method according to claim 22, wherein the androgenic compound is selected from the group consisting of oxandrolone, oxymetholone, stanozolol, stanozolone, danazol, pharmaceutically acceptable esters and salts thereof, and combinations of any of the foregoing.

24. The method according to claim 16, wherein said second dose of an estrogenic compound is administered after therapy of menopause has been effectively established.

25. The method according to claim 16, wherein said second dose of an estrogenic compound is administered between 2 weeks and 12 weeks after the first dose of an estrogenic compound.

26. The method according to claim 16, wherein said second dose of an estrogenic compound is administered between 4 weeks and 8 weeks after the first dose of an estrogenic compound.

27. The method according claim 16, wherein said first dose is continuously and uninterruptedly administered to said subject for a predetermined period of time and then said second dose is continuously and uninterruptedly administered to said subject.

28. The method according to claim 16, wherein said subject is human.

29. A method of treating hormonal deficiencies in a subject comprising:
administering a first dose of a therapeutic amount of an estrogenic compound to a subject; and
administering a second dose of a therapeutic amount of an estrogenic compound to the subject at a later time period, said second dose comprising a lower dosage of said therapeutic amount of an estrogenic compound than said first dose.

30. The method according to claim 29, wherein said estrogenic compound is a conjugated estrogen.

31. The method according to claim 29, wherein said first dose comprises 0.5 to 3 mg of an estrogenic compound.

32. The method according to claim 29, wherein said first dose comprises 0.6 to 1.25 mg of an estrogenic compound.

33. The method according to claim 29, wherein said estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

34. The method according to claim 29, further comprising administering a progestin in a daily dose.

35. The method according to claim 29, further comprising administering an androgen compound in a daily dose.

36. The method according to claim 35, wherein the androgenic compound is selected from the group consisting of oxandrolone, oxymetholone, stanozolol, stanozolone, danazol, pharmaceutically acceptable esters and salts thereof, and combinations of any of the foregoing.

37. The method according to claim 29, wherein said second dose of an estrogenic compound is administered between 2 weeks and 12 weeks after the first dose of an estrogenic compound.

38. The method according to claim 29, wherein said second dose of an estrogenic compound is administered between 4 weeks and 8 weeks after the first dose of an estrogenic compound.

39. The method according claim 29, wherein said first dose is continuously and uninterruptedly administered to said subject for a predetermined period of time and then said second dose is continuously and uninterruptedly administered to said subject.

40. The method according to claim 29, wherein said subject is human.

41. A method of treating vasomotor symptoms in a subject, said method comprising:
administering continuously and uninterruptedly for a first predetermined time period a first dose of an estrogenic compound to said subject; and
administering continuously and uninterruptedly for a second predetermined time period a second dose of an estrogenic compound to said subject.

42. The method according to claim 41, wherein said estrogenic compound is a conjugated estrogen.

43. The method according to claim 41, wherein said first dose comprises 0.5 to 3 mg of an estrogenic compound.

44. The method according to claim 41, wherein said first dose comprises 0.6 to 1.25 mg of an estrogenic compound.

45. The method according to claim 41, wherein said estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

46. The method according to claim 41, further comprising administering a progestin in a daily dose.

47. The method according to claim 41, further comprising administering an androgen compound in a daily dose.

48. The method according to claim 47, wherein the androgenic compound is selected from the group consisting of oxandrolone, oxymetholone, stanozolol, stanozolone, danazol, pharmaceutically acceptable esters and salts thereof, and combinations of any of the foregoing.

49. The method according to claim 41, wherein said second dose of an estrogenic compound is administered after therapy of the vasomotor symptoms has been effectively established.

50. The method according to claim 41, wherein said first predetermined time period for said first dose of an estrogenic compound is at least twelve weeks before the administration of said second dose of an estrogenic compound.

51. The method according to claim 41, wherein said first predetermined time period for said first dose of an estrogenic compound is between four to eight weeks before the administration of said second dose of an estrogenic compound.

52. The method according to claim 41, wherein said vasomotor symptoms are selected from the group of hot flashes, cold flashes, night sweats, day sweats, dry vagina, dry hair and skin, insomnia, bladder problems and moodiness.

53. The method according to claim 41, wherein said subject is human.

54. A method for treating a patient afflicted with vasomotor symptoms, comprising administering an estrogenic compound to said patient for at least two cycles of a cyclical dosing schedule, wherein the first cycle comprises a dosing period of 4 to 12 weeks, in which the estrogenic compound is administered daily, at a dose of 0.625 to 1.5 mg/day, followed by a second cycle comprising a dosing period that can last for an indeterminate period of time in which an estrogenic compound is administered daily, at a dose of 0.05 to 0.625 mg/day.

55. A method for treating a patient afflicted with vasomotor symptoms, comprising administering an estrogenic compound to said patient for at least two cycles of a cyclical dosing schedule, wherein the first cycle comprises a dosing period until such dosing has been shown to be effectively established, in which the estrogenic compound is administered daily, at a dose of 0.625 to 1.5 mg/day, followed by a second cycle comprising a dosing period that can last for an indeterminate period of time in which an estrogenic compound is administered daily, at a dose of 0.05 to 0.625 mg/day.
